(19) United States
(12) Patent Application Publication
(10) Pub. No.: US 2016/0222419 A1
(43) Pub. Date: Aug. 4, 2016

(54) GENETICALLY ENGINEERED SACCHAROMYCES CEREVISIAE AND YARROWIA LIPOLYTICA AND PRODUCTION OF FATTY ALCOHOLS

(71) Applicant: THE GOVERNORS OF THE UNIVERSITY OF ALBERTA, Edmonton (CA)

(72) Inventor: David Troy STUART, Edmonton (CA)

(73) Assignee: The Governors of the University of Alberta, Edmonton, AB (CA)

(21) Appl. No.: 15/022,844

(22) PCT Filed: Sep. 18, 2014
(86) PCT No.: PCT/US2014/056382

§ 371 Date: Mar. 17, 2016

Related U.S. Application Data
(60) Provisional application No. 61/879,453, filed on Sep. 18, 2013.

(51) Int. Cl. C12P 7/04; C12N 15/81; C12N 9/00; C12P 7/64; C12N 9/02; C12N 9/16

(52) U.S. Cl. CPC C12P 7/04; C12N 9/0008; C12N 9/16; C12N 9/93; C12P 7/64; C12N 15/81; C12Y 102/01084; C12Y 301/02; C12Y 602/01003

(57) ABSTRACT

Genetically engineered Saccharomyces cerevisiae and Yarrowia lipolytica for the production of fatty alcohols, including hexadecanol and octadecanol, are provided. The S. cerevisiae and Y. lipolytica can be genetically engineered to express or over-express a plurality of enzymes involved in fatty alcohol synthesis, such as acyl-CoA thioesterase, fatty acyl reductase, fatty acyl activating enzyme and fatty acyl synthetase, and can be further genetically engineered with a deficiency in one or more enzymes involved in fatty acid oxidation, such as fatty acyl-CoA oxidase. Methods of producing fatty alcohols, such as hexadecanol and octadecanol, by fermenting the genetically engineered S. cerevisiae and Y. lipolytica are also described.

### RELATED APPLICATIONS

This application claims priority under 35 U.S.C. §119(e) to U.S. Provisional Patent Application No. 61/879,453, filed Sep. 18, 2013, which is incorporated herein by reference.

### FIELD OF THE INVENTION

The present invention relates to the field of fatty alcohol production and, in particular, to the use of genetically engineered Saccharomyces cerevisiae and Yarrowia lipolytica to produce fatty alcohols.

### BACKGROUND OF THE INVENTION

Fatty alcohols are long chain aliphatic molecules with nonpolar lipophilic hydrocarbon chains and a polar hydrophilic hydroxyl moiety. The hydrocarbon chain lengths of naturally occurring fatty alcohols are highly variable ranging from 8 to 30 carbon atoms. Many organisms synthesize fatty alcohols in small amounts and these molecules are used as pheromones by insects [1], components of waxes that coat plants, insects and mammals [2], ether lipids that form the base of signaling molecules [3], and as components of storage lipids [4, 5]. Naturally occurring fatty alcohols are derived from fatty acid biosynthetic pathways and so most have even numbered carbon chains although some odd chain length alcohols have been identified in bacteria [6]. While the majority of naturally occurring fatty alcohols are saturated, some monounsaturated alcohols have been detected incorporated in wax esters and ether lipids [2].

The biosynthesis of fatty alcohols proceeds by the activation of fatty acids to acyl-CoA and subsequent reduction by fatty acyl reductase (FAR) activities that catalyze a four-electron reduction of acyl-CoA. The reaction proceeds through an aldehyde intermediate although in many cases no free aldehyde is released [7]. Most alcohol forming FAR activities use reducing equivalents from NADPH [8], however an NADH dependent aldehyde forming FAR activity has been identified in the algae Botryococcus brauni [9]. The nature of the available pool of fatty acids is a major determinant of the type of fatty alcohols that will be produced by any organism.

The relatively low abundance of naturally occurring unsaturated fatty alcohols suggests that the FAR catalyzed reaction takes place before the cells can convert the saturated acyl-CoA to an unsaturated chain. However, this may also reflect the substrate specificity of the FAR enzyme catalyzing the reaction. CfFAR1 from the copepod Calanus finmarchicus displays rigid specificity for saturated acyl-CoA substrates while CfFAR3 displays excellent activity toward monounsaturated C18:1-CoA [10]. Additionally, FAR enzymes display some preference for the carbon chain length of the substrate. When expressed in yeast where C18:1 is the most abundant fatty acid, the Arabidopsis thaliana FAR1, FAR2, FAR4, and FAR5 enzymes yielded dramatically different profiles of fatty alcohols produced. FAR5 yielded primarily C18:0 octadecanol whereas FAR3 yielded almost exclusively C26:0 alcohol [11].

Most alcohol forming FAR enzymes are integral membrane proteins that have a hydrophobic sequence targeting them to the endoplasmic reticulum, Golgi apparatus, or peroxisomes. In contrast aldehyde forming FAR enzymes are cytoplasmic or peripheral membrane proteins [9, 10, 12]. The localization of the enzyme likely reflects its function. Whereas A. thaliana FAR enzymes are largely associated with the endoplasmic reticulum and wax ester synthesis, the mouse FAR1 and FAR2 are localized to peroxisomes for the synthesis of ether lipids [13, 14].

Fatty alcohols have a variety of industrial applications. Owing to the amphiphilic nature of fatty alcohols that results from the combination of a long nonpolar acyl chain and a polar hydrophilic hydroxyl these molecules can act as surfactants and a significant portion of fatty alcohols produced are used for this purpose [15]. Their chemical character allows fatty alcohols to orient themselves at interfaces for use as emulsifiers and emollients in the cosmetic industry. Additionally, they are used as additives in lubricant, surfactants and detergent formulations as polythoxylates. Some short chain alcohols are used as flavor and fragrance compounds. They find broad application as platform chemicals since the alcohol moiety is vulnerable to a variety of chemical modifications, and they can be used to form polymers [16]. Additionally, fatty alcohols can also be used as fuel additives [17].

Fatty alcohols in their free form are very rare in nature with most being derived from waxes into which they are incorporated [2]. Fatty alcohols can be produced by hydrolysis or trans-esterification of triglycerides derived from vegetable oils followed by hydrogenation to produce alcohols. Indeed this process makes up the primary source of fatty alcohols employed in industrial and commercial applications [17]. Fatty alcohols can also be chemically synthesized from petrochemical feedstock through oligomerization of ethylene followed by oxidation [17].

Increasing costs associated with the production of petroleum products, concerns about food oil security, and environmental impacts of replacing rainforest with palm oil plantations have increased interest in development of microbial systems that can catalyze the conversion of low value carbohydrate feedstock into high value lipid products.

Currently the primary source of the widely used C16 and C18 hexadecanol and octadecanol is reduction of palm oil [17]. Similarly C12 and C14 dodecanol and teradecanol are derived from palm kernel and coconut oil [17]. These are food oils of choice for much of the world [18] and increased industrial demand has lead to extensive deforestation of rainforest in Asia with resulting loss of biodiversity and replacement of forest with palm plantations [19, 20]. Considering the broad use of fatty alcohols, and the desire to minimize deforestation microbial biosynthesis of fatty alcohols is a desirable goal.

The biosynthesis of C12/C14 and C16/C18 alcohols through engineering the expression of FAR and fatty acid activating enzymes in E. coli and cyanobacteria Synechocystis sp has been reported [21-23]. Routes to the synthesis of fatty alcohol species using carboxylic acid reductase (CAR) have also been described [24].

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In accordance with one aspect, the invention relates to an isolated Saccharomyces cerevisiae and Yarrowia lipolytica cells genetically engineered to produce one or more fatty alcohols, the S. cerevisiae and Y. lipolytica cells genetically engineered to express or overexpress two or more enzymes involved in fatty alcohol synthesis and further genetically engineered to be deficient in at least one enzyme involved in fatty acid oxidation.

In accordance with another aspect, the invention relates to an isolated Saccharomyces cerevisiae cell genetically engineered to produce one or more fatty alcohols, the S. cerevisiae cell genetically engineered to: i) express an acyl-CoA thioesterase; ii) express a fatty acyl reductase, and iii) (a) express a fatty acyl activating enzyme, or (b) lack a fatty acyl-CoA oxidase, or (c) both express a fatty acyl activating enzyme and lack a fatty acyl-CoA oxidase.

In accordance with another aspect, the invention relates to an isolated S. cerevisiae and Y. lipolytica cells genetically engineered to produce one or more fatty alcohols, the S. cerevisiae and Y. lipolytica cells genetically engineered to express an acyl-CoA thioesterase from E. coli, a fatty acyl reductase from Mus musculus and an endogenous fatty acyl activating enzyme, to over-express an endogenous fatty acyl synthetase and to lack an endogenous fatty acyl-CoA oxidase. Moreover, these enzymes can be expressed from a constitutive or heterologous promoter.

In accordance with another aspect, the isolated S. cerevisiae and Y. lipolytica cells are genetically engineered to contain mutations or deletions in several genes, including, but not limited to SNF2, ADH1 and ACB1. In other embodiments, genes may also be added to increase fatty alcohol synthesis such as, but not limited to ALD6 and ACS1.

In accordance with another aspect, the invention relates to an isolated nucleic acid comprising a first nucleotide sequence encoding an acyl-CoA thioesterase, a second nucleotide sequence encoding a fatty acyl reductase and a third nucleotide sequence encoding a fatty acyl activating enzyme.

In accordance with another aspect, the invention relates to a vector comprising the isolated nucleic acid as described above.

In accordance with another aspect, the invention relates to isolated S. cerevisae and Y. lipolytica cells comprising either of the isolated nucleic acid or the vector described above.

In accordance with another aspect, the invention relates to method of producing one or more fatty alcohols comprising culturing the isolated S. cerevisiae and Y. lipolytica cells described above and isolating the one or more fatty alcohols.

In accordance with another aspect, the invention relates to a method of producing fatty alcohols comprising hexadecanol and octadecanol, the method culturing an isolated S. cerevisiae and Y. lipolytica cells genetically engineered to express an acyl-CoA thioesterase from E. coli, a fatty acyl reductase from Mus musculus and an endogenous fatty acyl activating enzyme, to over-express an endogenous fatty acyl synthetase and to lack an endogenous fatty acyl-CoA oxidase in a medium comprising glucose and isolating the one or more fatty alcohols.

### BRIEF DESCRIPTION OF THE DRAWINGS

These and other features of the invention will become more apparent in the following detailed description in which reference is made to the appended drawings.

FIG. 1. Expression of Fatty alcohol synthesizing enzymes in S. cerevisiae. Protein extracts from 3 independent colonies were probed with anti-MYC ascites fluid for the presence of (A) murine FAR1, (B) E. gracilis FAR1, (C) E. coli tesA, (D) S. cerevisiae Tes1. Strains harboring an empty expression vector were probed as a negative control (vector). As a positive control for the antibody binding and western blotting an extract containing a MYC tagged Erg10 was included in each blot.

FIG. 2. Expression of E. coli tesA increases fatty acid accumulation in S. cerevisiae. S. cerevisiae harboring an empty vector (grey bars), a vector overproducing TES1 (open bars) or a vector overproducing E. coli TesA (striped bars) were cultured in selective medium and lipids were extracted and measured by gas chromatography. Fatty acid concentrations are expressed as mg/gram wet weight of cells. The means of three independent experiments are shown, error bars indicate standard deviation for the three independent trials.

FIG. 3. Production of hexadecanol and octadecanol in S. cerevisiae. Chromatograms from GC-FID analysis of lipid from S. cerevisiae harboring an empty expression vector (A) or the pALCm expression vector (B). Peaks corresponding to methyl esters (ME) and fatty alcohols (A1c) are indicated by arrows. C19:0 was included as an internal standard. The Peak corresponding to MSTFA derivatized C16:0 alcohol was characterized by mass spectrometry to confirm its identity (C).

FIG. 4. Engineering the fatty acid biosynthesis pathway increases fatty acid and fatty alcohol accumulation in S. cerevisiae. (A) GC-FID analysis of lipids in S. cerevisiae harboring the endogenous FAS1 or a PPGK1-FAS1 a fusion of the FAS1 open reading frame to the PGK1 promoter. Fatty acid concentrations are expressed as mg/g cells wet weight. Data shown are the means of three independent experiments. Errors bars indicate standard deviation. (B) GC-FID analysis of S. cerevisiae harboring the pALCm plasmid and the indicated additional genetic modifications. Fatty alcohol concentrations are expressed as mg/g wet weight of cells. The mean values from two independent trials are shown. (C) GC-FID analysis of fatty acids from the same strains shown in panel B. Fatty acid concentrations are expressed as mg/g wet weight of cells. Data shown are the mean values from two independent trials.

FIG. 5. Reduced nitrogen availability increases both fatty acid and fatty alcohol production in engineered S. cerevisiae. GC-FID analysis of lipids from S. cerevisiae DSY2003 (pox1::KanMX4 fas1::PPGK1-FAS1) harboring the expression plasmid pALCm. Cells were cultured in synthetic complete medium containing either 5 g/L ammonium sulfate (+ Nitrogen, light grey bars) or with no added ammonium sulfate (− Nitrogen, dark grey bars). Fatty alcohol (A) and fatty acid (B) concentrations are expressed as mg/g wet weight of cells. Data shown are the means from two independent experiments.

FIG. 6. Fatty alcohol production in S. cerevisiae fed batch culture. GC-FID analysis of fatty alcohol (A) and fatty acids (B) at the indicated times during a fed batch fermentation of strain DSY2003 harboring pALCm. The data shown are from a single 4 litre fermentation. The growth of the culture is indicated by increasing OD₆₀₀ values (C) and the culture was fed with glucose at the times indicated by the arrows in panel C.

FIG. 7. Engineered yeast produce C16 and C18 alcohols. Mutations in SNF2 and FAR1 increase yields. Expression of Arabadopsis thaliana FAR5 in S. cerviseae leads to production of exclusively monounsaturated octadecanol compared with the mixed hexadecanol and octadecanol synthesized by mFAR1. Additionally, we determined that deletion of the ACB1 gene could increase the proportion of octadecanol synthesized. The Acb1 protein is involved in transport of octadecanoic acid and its deletion increases the cellular pools of octadecanoic acid available for conversion to octadecanol. Note that ACB1 deletion increases the proportion of octadecanol produced in FAR1 expressing strains and increases the abundance of octadecanol in FAR5 expressing strains.

FIG. 8. Yeast expressing FAR5 make only C18 alcohol and ACB1 deletion increases the production of C18 alcohol. Mutation of the gene for SNF2, which encodes a chromatin-remodeling enzyme, increases the production of fatty alcohols in FAR1 expressing strains. This mutant was identified in a screen for strains with increases lipid production. The FAR1 expressing strain was mutagenized with ultraviolet light and surviving strains were layered on top of and centrifuged into a density gradient. More buoyant cells (with higher lipid content) floated near the top of the gradient. Several of the more buoyant strains were found to be lipid and fatty alcohol over producers and had mutations in the SNF2 gene. Truncation of FAR1 also increases fatty alcohol production. 48 amino acids predicted to encode a membrane anchoring domain in FAR1 were removed to allow FAR1 to function in the cytoplasm. This truncation improves production of fatty alcohols likely by increasing access of Far1 to the cytoplasm fatty acid pool.

FIG. 9. C16 and C18 alcohols can be made from hydrolysates of softwood or wheat. The fatty alcohol producing strains can use cellulosic hydrolysates as feedstock. Hydrolysates of softwood or wheat straw produced by dilute acid treatment were used to provide a carbon source with a sugar content of 40 g/L. The hydrolysates were supplemented with urea to provide nitrogen. Yields of hexadecanol and octadecanol are lower than observed with sugar as a substrate in part owing to inhibitors of cell growth present in the hydrolysates and owing to the ability of the production strain to effectively utilize xylose and arabinose which account for some of the sugars in the hydrolysates. The strain can and will be adapted to tolerate inhibitors in the hydrolysates and will be engineered to effectively ferment xylose and arabinose as well as the glucose in the hydrolysates.

### DETAILED DESCRIPTION OF THE INVENTION

The present invention relates to the genetic engineering of Saccharomyces cerviseae for the production of fatty alcohols, including, but not limited to hexadecanol and octadecanol. In particular, it has been discovered that genetically engineering S. cerevisiae to express or over-express a plurality of enzymes involved in fatty alcohol synthesis allows production of fatty alcohols in S. cerevisiae. In addition, genetic engineering of S. cerevisiae to express a plurality of enzymes involved in fatty alcohol synthesis in combination with a deficiency in one or more enzymes involved in fatty acid oxidation may lead to increased yields of fatty alcohols in some embodiments.

Accordingly, in some embodiments, the invention relates to S. cerevisiae genetically engineered to express or overexpress two or more enzymes involved in fatty alcohol synthesis and further genetically engineered to be deficient in at least one enzyme involved in fatty acid oxidation.

Certain embodiments of the invention also relate to methods of producing fatty alcohols by culturing the genetically engineered S. cerevisiae described herein.

The two or more enzymes involved in fatty alcohol synthesis may comprise, for example, acyl-CoA thioesterase and fatty acyl reductase. In certain embodiment, the acyl-CoA thioesterase may be derived from a bacterial source, for example, from E. coli. In some embodiments, the E. coli acyl-CoA thioesterase may be mutated or truncated. In certain embodiments, the fatty acyl reductase may be derived from a mammalian source, for example, from a rodent such as Mus musculus, or from plant sources including but not limited to Arabadopsis thaliana, or from single cell organism such as Euglena gracilis. The fatty acyl reductase may also be mutated or truncated to increase fatty alcohol synthesis.

In certain embodiments, the enzymes involved in fatty alcohol synthesis may comprise, for example, acyl-CoA thioesterase, fatty acyl reductase and fatty acyl activating enzyme. The fatty acyl activating enzyme may be, for example, an endogenous fatty acyl activating enzyme. Fatty acyl activating enzymes derived from other sources, including bacterial, plant, single cell organisms, or mammalian, are also contemplated in some embodiments.

In certain embodiments, the enzymes involved in fatty alcohol synthesis may comprise, for example, acyl-CoA thioesterase, fatty acyl reductase, fatty acyl activating enzyme and fatty acyl synthetase. The fatty acyl synthetase may be, for example, an endogenous fatty acyl synthetase. Fatty acyl synthase enzymes derived from other sources, including bacterial, plant, single cell organisms, or mammalian, are also contemplated in some embodiments. In certain embodiments, in which the fatty acyl synthase is an endogenous fatty acyl synthase, the S. cerevisiae is genetically engineered to place expression of the endogenous fatty acyl synthase under control of a heterologous promoter in order to reduce feedback inhibition of fatty acyl synthase expression. It also contemplated that the other genes involved in fatty alcohol synthesis my also be under the control of a heterologous promoter.

In further embodiments, other exogenous enzymes may be expressed in S. cerevisiae to increase the yield of fatty alcohols. For example, the addition of malonyl-CoA synthetase from A. thaliana, and glyceraldehyde-3-phosphate dehydrogenase from Streptomyces mutans can increase the cellular concentration of malonyl-CoA (substrate for acyl-CoA synthesis) and NADPH (cofactor for acyl-CoA synthesis) respectively. Other enzymes that may be manipulated to increase fatty alcohol yield include the up-regulation of the fatty acid synthase subunits Fas1 and Fas2.

In certain embodiments, one or more of the nucleic acids used to express the acyl-CoA thioesterase, fatty acyl reductase, fatty acyl activating enzyme and fatty acyl synthetase may be codon optimized to improve expression in S. cerevisiae. In certain embodiments, each of the nucleic acids used to express the acyl-CoA thioesterase, fatty acyl reductase, fatty acyl activating enzyme and fatty acyl synthetase may be under the control of respective constitutive promoters so that production of fatty alcohols by fermentation of the S. cerevisiae does not require addition of specific compounds to induce expression of these enzymes.

In certain embodiments, the enzymes involved in fatty acid oxidation comprise, for example, fatty acyl-CoA oxidase (POX1 gene).

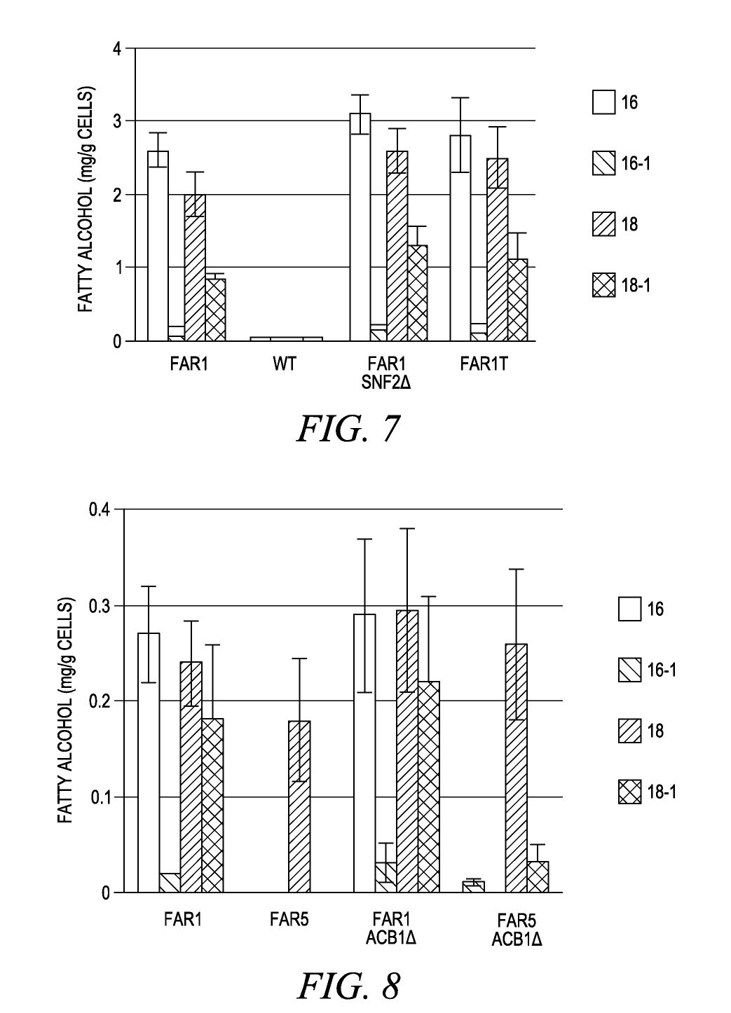

In other embodiments, it may be advantageous to mutate or delete various genes in order to increase fatty alcohol production. For example, a mutation in the SNF2 gene may increase fatty alcohol production (see FIGS. 7-8). The mutation isolated was a null mutation creating a frame shift in the coding sequence of the gene. A complete deletion of SNF2 has the same effect as the null frame shifting mutation

In another embodiment, it may be possible to increase the yield of fatty alcohol production (e.g. hexadecanol and octadecanol) through the mutation or deletion of genes encoding acyl binding proteins, such as, but limited to ACB1, which promotes the degradation of C18-CoA (see FIG. 8).

As is known in the art, different acyl-CoA thioesterase and fatty acyl reductase enzymes can have different specificities with respect to the type of fatty acid they use as a substrate and/or the type of fatty alcohol they produce (11). Accordingly, in certain embodiments, the acyl-CoA thioesterase and/or fatty acyl reductase for genetic engineering of the S. cerevisiae are selected depending on what fatty alcohol(s) are required in the end product (see FIG. 7 for example).

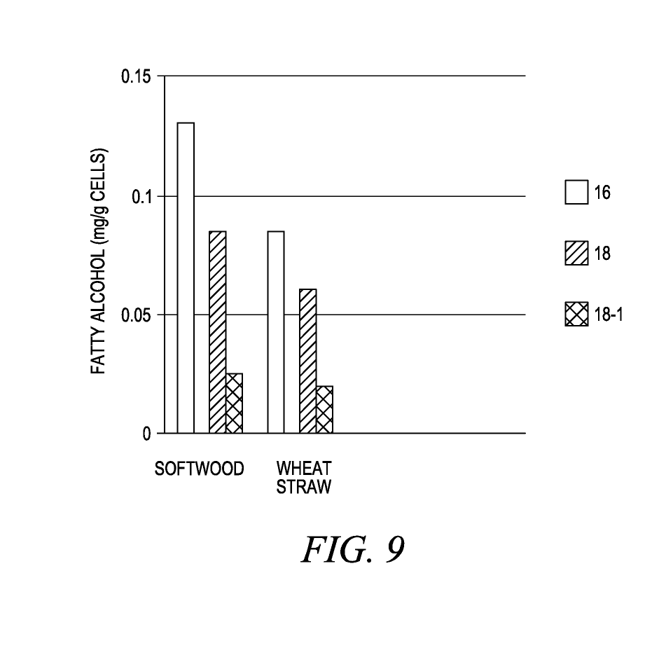

In certain embodiments, the S. cerevisiae strain selected for genetic engineering in accordance with the invention is one that is capable of fermenting sugars from lignocellulosic hydrolysates. In some embodiments, the S. cerevisiae strain selected for genetic engineering in accordance with the invention is one that is capable of fermenting sugars from lignocellulosic hydrolysates and is resistant to the most common inhibitory compounds present in such lignocellulosic hydrolysates. In some embodiments, the S. cerevisiae strain selected for genetic engineering in accordance with the invention is one that is capable of fermenting both glucose and xylose from lignocellulosic hydrolysates (see FIG. 9). Such strains are known in the art (36, 37, 38).

In certain embodiments, fermentation of the genetically engineered S. cerevisiae described above in a suitable medium allows for high-level production of fatty alcohols, for example, greater than 100 mg/L in 168 hours, for example, at least 110 mg/L, at least 120 mg/L, at least 130 mg/L, at least 140 mg/L, at least 150 mg/L, at least 160 mg/L, at least 170 mg/L, in 168 hours, or any amount there between. In certain embodiments, fermentation of the genetically engineered S. cerevisiae described above in a suitable medium allows for production of 100 mg/L or greater of fatty alcohols in 72 hours, for example, 110 mg/L or greater, 120 mg/L or greater, 130 mg/L or greater, 140 mg/L or greater, 150 mg/L or greater, 160 mg/L or greater, 170 mg/L or greater, in 72 hours, or any amount there between.

In addition to S. cerevisiae, in other embodiments, the oleaginous yeast strain Yarrowia lipolylica is used to increase yields of fatty alcohols. Y. lipolylica is capable of fermenting sugars from lignocellulosic hydrolysates and can be advantageous in some situations due to its high rate of fatty acid accumulation and the usage of a wide variety of sugars, glycerol and acetic acid as carbon sources.

In some embodiments, Y. lipolytica strains can be genetically engineered to express or overexpress two or more enzymes involved in fatty alcohol synthesis such as described above with S. cerevisiae strains. For example, in some Y. lipolytica strains, the enzymes involved in fatty alcohol synthesis may comprise, for example, acyl-CoA thioesterase, fatty acyl reductase, fatty acyl activating enzyme and fatty acyl synthetase or any combination thereof. These enzymes may be, for example, endogenous or can including bacterial, plant, single cell organisms, or mammalian sources.

In certain embodiments, one or more of the nucleic acids used to express the acyl-CoA thioesterase, fatty acyl reductase, fatty acyl activating enzyme and fatty acyl synthetase may be codon optimized to improve expression in Y. lipolytica. In certain embodiments, each of the nucleic acids used to express the acyl-CoA thioesterase, fatty acyl reductase, fatty acyl activating enzyme and fatty acyl synthetase may be under the control of respective constitutive promoters so that production of fatty alcohols by fermentation of the Y. lipolytica does not require addition of specific compounds to induce expression of these enzymes.

In still further embodiments, each of the nucleic acids used to express the acyl-CoA thioesterase, fatty acyl reductase, fatty acyl activating enzyme and fatty acyl synthetase may be under the control of respective heterologous promoters. As a non-limiting example, where the fatty acyl synthase is an endogenous fatty acyl synthase, the Y. lipolytica is genetically engineered to place expression of the endogenous fatty acyl synthase under control of a heterologous promoter in order to reduce feedback inhibition of fatty acyl synthase expression.

In some embodiments, Y. lipolytica strains express the FAR1 gene of M. mus and the tesA gene of E. coli.

In further embodiments, Y. lipolytica strains expressing the FAR1 gene of A. thaliana and the tesA gene of E. coli, and may be deficient in another gene product, or have further genes added to increase the yield of fatty alcohols. For example, the flow of carbon may be redirected from the production of ethanol to the production of acetyl-CoA for fatty acyl-CoA synthesis. This can be accomplished, for example, by the deletion of the ADH1 gene and addition of genes encoding ALD6 and a hyperactive ACS1 to convert acetaldehyde to acetyl-CoA as substrate for fatty acyl-CoA synthesis. Considering that Ald6 uses NADP as a cofactor this will lead to an increase in NADPH, which is required for fatty alcohol synthesis.

In situations where NADPH is limiting production of fatty alcohol synthesis, a further increase in NADPH can be achieved through expression of xpkA (xylose-5-phosphate kinase) and ack (acetate kinase) from Aspergillus nidulans. These enzymes create a heterologous phosphoketolase pathway leading to elevated NADPH production.

Moreover, some embodiments of engineered Y. lipolytica strains can be further modified by the introduction of a mutation to, or a deletion of the SNF2 gene (see FIGS. 7-8). The SNF2 gene encodes a chromatin remodeling protein and is involved in the transcriptional regulation of the fatty alcohol synthesis genes. Such a mutation can increase fatty acid synthesis.

In other embodiments, a codon optimized xylose isomerase derived from Piromyces sp. can be inserted in the yeast strain to increase their ability to use the xylose in cellulosic hydrolysates. The techniques used in the manipulation of recombinant DNA are well known to those of ordinary skill in the art.

In certain embodiments, the Y. lipolytica strain selected for genetic engineering in accordance with the invention is one that is capable of fermenting sugars from lignocellulosic hydrolysates. In some embodiments, the Y. lipolytica strain selected for genetic engineering in accordance with the invention is one that is capable of fermenting sugars from lignocellulosic hydrolysates and is resistant to the most common inhibitory compounds present in such lignocellulosic hydrolysates. In some embodiments, the Y. lipolytica strain selected for genetic engineering in accordance with the invention is one that is capable of fermenting both glucose and xylose from lignocellulosic hydrolysates.

In certain embodiments, fermentation of the genetically engineered Y. lipolytica described above in a suitable medium allows for high-level production of fatty alcohols, for example, greater than 100 mg/L in 168 hours, for example, at least 110 mg/L, at least 120 mg/L, at least 130 mg/L, at least 140 mg/L, at least 150 mg/L, at least 160 mg/L, at least 170 mg/L, in 168 hours, or any amount there between. In certain embodiments, fermentation of the genetically engineered Y. Lipolytica described above in a suitable medium allows for production of 100 mg/L or greater of fatty alcohols in 72 hours, for example, 110 mg/L or greater, 120 mg/L or greater, 130 mg/L or greater, 140 mg/L or greater, 150 mg/L or greater, 160 mg/L or greater, 170 mg/L or greater, in 72 hours, or any amount there between.

Conditions that are relevant to optimal growth of yeast cultures include, but are not limited to, fermentation temperature, pH, inoculation density and feeding schedules. For example, yeast cultures may be fermented at 30° C. in a synthetic medium supplemented with urea to provide a nitrogen source and a dissolved oxygen content of 30%. Glycerol and cellulosic hydrolysates can be used as a carbon feed stock for fatty alcohol production. The production of cellulosic hydrolysates is well known in the art. Dilute acid hydrolysis is one standard method for the production of cellulosic hydrolysates.

In addition, enzymes used in various embodiments may include those sharing a sequence identity or substantial sequence identity to those enzymes listed herein.

As used herein, “sequence identity” or “identity” in the context of two protein or peptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

The term “substantial identity” in the context of a proteins or peptide indicates that a proteins or peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### DEFINITIONS

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

The term “plurality” as used herein means more than one, for example, two or more, three or more, four or more, and the like.

“Naturally occurring,” as used herein, as applied to an object, refers to the fact that an object can be found in nature. For example, an organism, or a polypeptide or polynucleotide sequence that is present in an organism that can be isolated from a source in nature and which has not been intentionally modified by man in the laboratory is naturally occurring.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The use of the word “a” or “an” when used herein in conjunction with the term “comprising” may mean “one,” but it is also consistent with the meaning of “one or more.” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method or composition of the invention, and vice versa. Furthermore, compositions and kits of the invention can be used to achieve methods of the invention. To gain a better understanding of the invention described herein, the following examples are set forth. It will be understood that these examples are intended to describe illustrative embodiments of the invention and are not intended to limit the scope of the invention in any way.

### EXAMPLES

### Overview

Background:

Fatty alcohols are important components of a vast array of surfactants, lubricants, detergents, pharmaceuticals and cosmetics. C16 and C18 alcohols are rare in nature but can be synthesized from petroleum or from palm oil. However, the rising price of oil and the impact that expanding palm plantations have on biodiversity and diversion of food oils to industrial use has motivated increased efforts to develop microbial platforms for the biosynthesis of these valuable chemicals. We have investigated the potential to produce fatty alcohols in the yeast Saccharomyces cerevisiae owing to this organism's robust growth, genetic manipulability, and potential for the use of cellulosic hydrolysates as feedstock.

Results:

We engineered S. cerevisiae to produce C16 and C18 alcohols through introduction of a thioesterase activity from E. coli and a fatty acyl reductase from mouse, additionally a native acyl-CoA synthetase activity encoded by the FAA3 gene was over produced. The strain was further engineered by over expression of fatty acyl synthetase encoded by FAS1 and deletion of acyl-CoA oxidase encoded by POX1. In fed batch fermentation in rich medium this strain produced 194 mg/L of C16/C18 alcohols in 72 hours.

Conclusions:

Through engineering both pathways for fatty alcohol synthesis and degradation this work achieved C16 and C18 alcohol production in yeast without the use of inducing sugars or agents whose cost would be prohibitive for an industrial application. We show that yeast is a potential platform for industrial scale production of C16 and C18 fatty alcohols.

### Abbreviations:

GC Gas chromatography, GC-FID Gas chromatography with Flame Ionization Detector, FAR Fatty Acyl Reductase, NADH Nicotinamide Adenine Dinucleotide, NADPH Nicotinamide Adenine Dinucleotide Phosphate, rpm revolutions per minute, MSTFA Methanolic HCL, N-methyl-N-(trimethylsiylyl) trifluoroacetamide, CoA Coenzyme A, CAR Carboxylic Acid Reductase, DTT Dithiothreitol, SDS Sodium Dodecalsulfate.

### Example 1

The yeast Saccharomyces cerevisiae is an industrial workhorse for the production of ethanol and a variety of chemicals and small molecules. [25]. Yeast has great potential as a chassis for the production of lipid based compounds as some oleaginous species are capable of accumulating up to 70% of their dry weight as lipid [26]. Here we demonstrate the production of both saturated and monounsaturated hexadecanol and octadecanol by an engineered strain of S. cerevisiae. The high productivity of this strain (2.8 mg/L/Hr) and the robust nature of yeast offers the potential for microbial biosynthesis of hexadecanol and octadecanol at an industrial scale. While this manuscript was being revised synthesis of fatty alcohol was reported in S. cerevisiae however the reported yields were lower than obtained in this work [27]. The focus of this investigation was to determine whether S. cerevisiae could be developed as a potential platform for the industrial production of hexadecanol and octadecanol.

Results:

Fatty alcohol synthesizing enzymes can be expressed in S. cerevisiae. Fatty alcohols can be synthesized from fatty acids by activation of the fatty acid to an acyl-CoA and subsequent reduction to an alcohol. To determine whether fatty alcohols could be effectively produced by S. cerevisiae we introduced an expression vector harboring genes for an acyl-CoA thioesterase from either E. coli (tesA), or S. cerevisiae (TES1), a fatty acyl activating enzyme (FAA3) from S. cerevisiae and a fatty acyl reductase from either Mus musculus (mFAR1) or Euglena gracilis (egFAR1). Because tesA, mFAR1, and egFAR1 were heterologous enzymes the open reading frames were altered to include a 10 amino acid epitope tag recognized by the cMYC antibody. This allowed detection of the proteins by Western blotting to confirm expression. Additionally, a truncated version of tesA lacking its signal sequence was used since expression of this variant has previously been shown to result in elevated accumulation of fatty acids and to bypass feedback inhibition of fatty acyl-CoA on fatty acid synthesis [28].

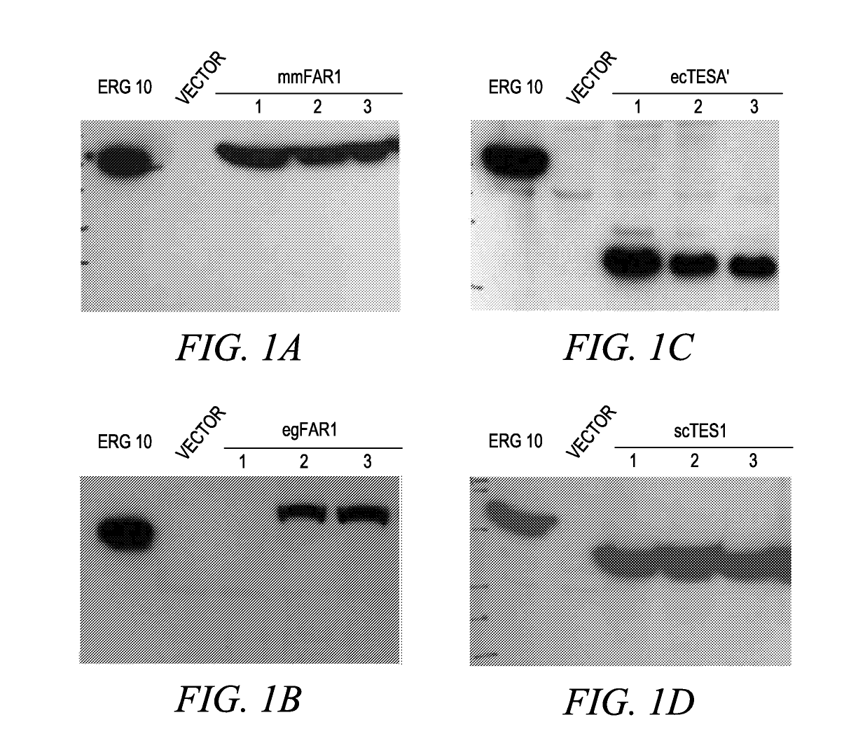

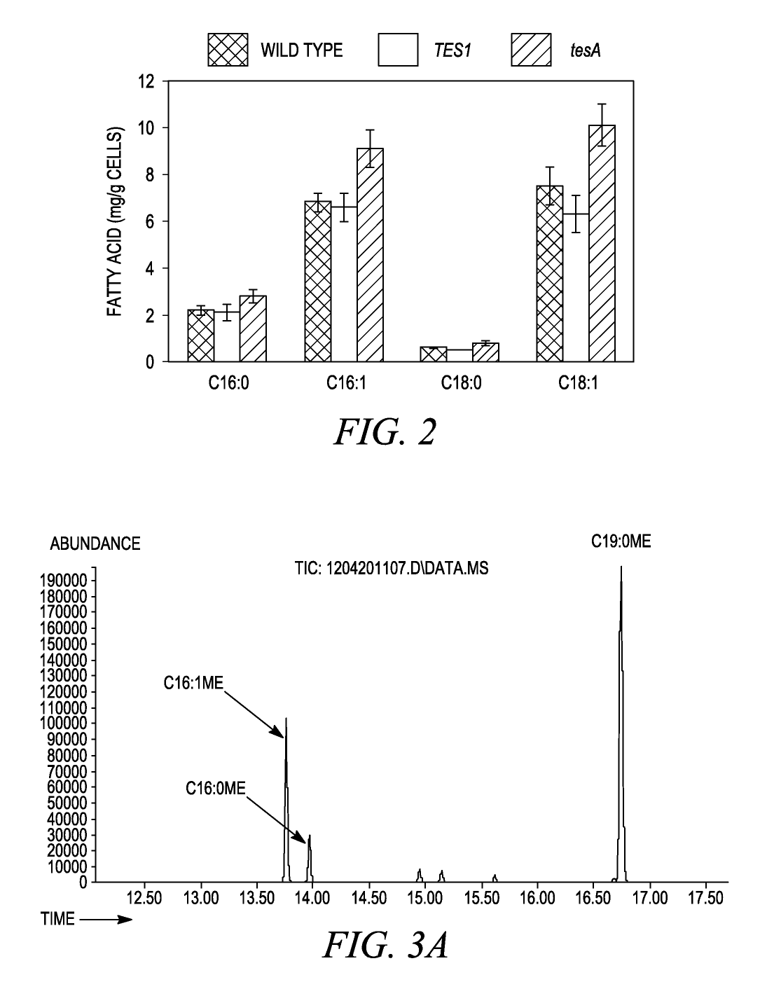

Western blotting extracts from candidate strains demonstrated that Tes1, tesA, mFar1, and egFAR1 were produced in the yeast strains (FIG. 1; A-D). The thioesterase was included to determine whether its expression would increase fatty acid availability as substrate for fatty alcohol synthesis. Over expression of TES1 had little discernable effect on the abundance of C16:0, C16:1, C18:0 or C18:1 fatty acids (FIG. 2). In contrast expression of a truncated form of E. coli tesA, lacking its leader sequence increased the cellular concentration of the most abundant fatty acids. C16:0 concentration increased by 22.5% while C16:1 increased by 34%, C18:0 and C18:1 were increased by 33% and 35% respectively (FIG. 2).

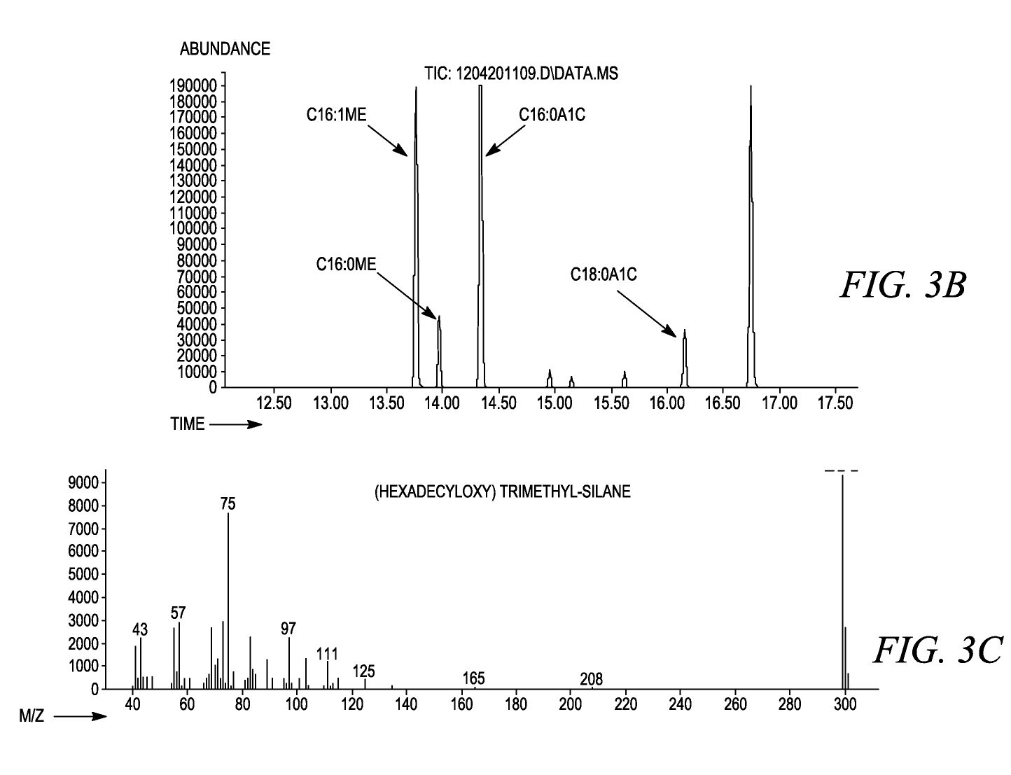

Synthesis of hexadecanol and octadecanol by engineered S. cerevisiae. To achieve conversion of fatty acids to fatty alcohols the fatty acyl-activating enzyme Faa3 was over produced in conjunction with tesA and with fatty acyl reductase from either Euglena gracilis or Mus musculus. Cell extracts were spiked with nonadecylic acid (C19) as an internal standard and the extracted lipids were analyzed by gas chromatography (FIG. 3). No fatty alcohols could be detected in a strain harboring the empty expression vector (FIG. 3A). In contrast, the combined expression of tesA, FAA3 and mFAR1 from plasmid pALC195m resulted in the production of both hexadecanol and octadecanol (FIG. 3B & Table 4). In two experiments with two independent S. cerevisiae strains yields of 1.64 mg/L and 1.72 mg/L hexadecanol were obtained in a 48 hour fermentation period corresponding to 0.23 mg/L/OD600 and 0.22 mg/L/OD600 (FIG. 3B & Table 4). Very little fatty alcohol synthesis was detected in strains expressing tesA, FAA3 and egFAR1 (0.01 mg/L octadecanol) (Table 4). The identity of the peak corresponding to hexadecanol was confirmed by mass-spectrometry (FIG. 3C). Thus the combined expression of acyl-thioesterase, fatty acyl activating enzyme and fatty acyl reductase can yield the synthesis of fatty alcohols in yeast. Interestingly, although C8:1 and C16:1 are the most abundant fatty acids in this yeast C16:0 and C18:0 were the most abundantly produced fatty alcohols. This may reflect the specificity of the mouse fatty acyl reductase.

Over expression of fatty acyl synthetase FAS1 increases fatty alcohol production in yeast. Fatty alcohols are synthesized from fatty acids and their CoA derivatives. Fatty acid synthesis is largely driven by the activity of the enzyme fatty acyl synthetase. In S. cerevisiae the FAS1 and FAS2 genes encode the 3 and a subunits of this enzyme. Fas1 and Fas2 function in a stoichiometric relationship and FAS2 gene expression is regulated in part by the Fas1 protein abundance such that an increase in Fas1 triggers increased transcription of FAS2 [29, 30].

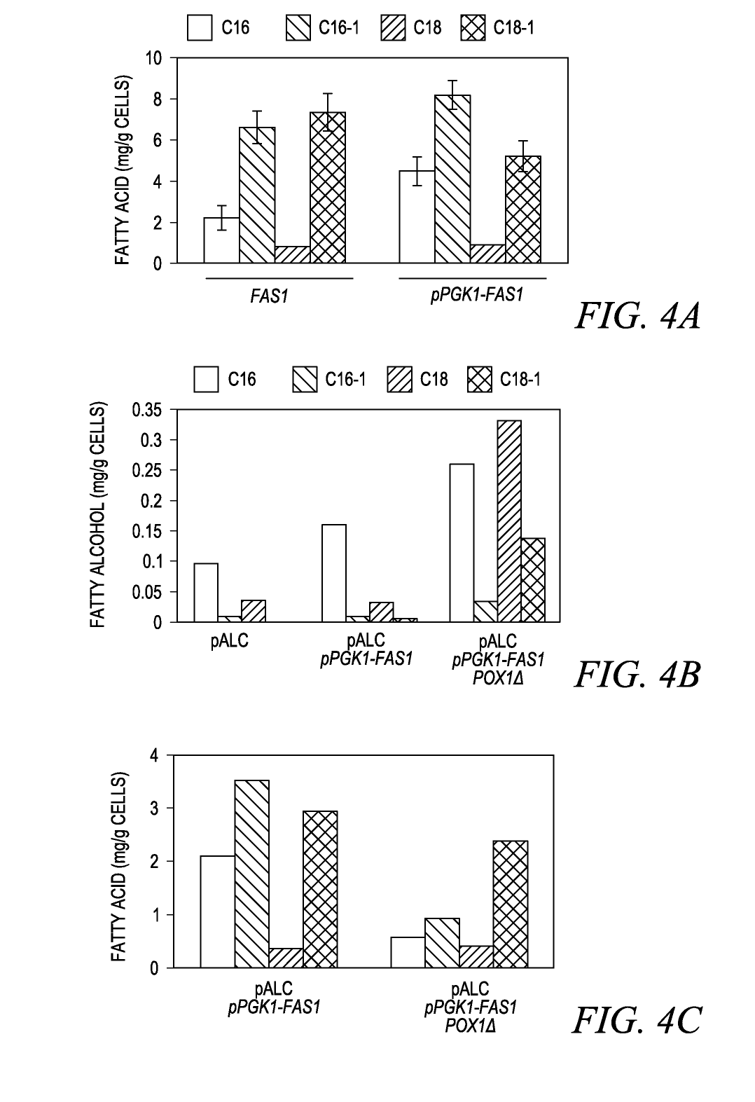

To increase carbon flux toward fatty acid synthesis and decouple FAS1 expression from feedback inhibition induced by the presence of saturated fatty acids [31], we replaced the endogenous FAS1 promoter with the promoter from the PGK1 gene. The PGK1 gene is highly expressed in cells growing in the presence of glucose and does not display regulation by fatty acids. The increased transcription of FAS1 yielded an increase in the cellular accumulation of C16:0 and C16:1 fatty acids. C16:0 increased from 2.21 mg/g cells to 4.48 mg/g cells and C16:1 increased from 6.62 mg/g cells to 8.19 mg/g cells (FIG. 4A). In contrast upregulation of FAS1 in this strain yielded no significant increase C18:0 and C18:1 (FIG. 4A). Cells overexpressing FAS1 and harbouring the pALC195m plasmid expressing tesA. FAA3 and mFAR1 displayed an increased accumulation of fatty alcohols. Hexadecanol concentrations of 0.16 mg/g-wet weight of cells were found in FAS1 over expressing strains compared to 0.096 mg/g cells in the control strain (FIG. 4B). Octadecanol concentrations did not display a significant increase possibly reflecting the lack of increase in octadecanoic acid accumulated in the FAS1 over expressing cells.

The size of the intracellular fatty acid pool is a key determinant for fatty alcohol production and so we investigated other avenues of engineering increased fatty acid synthesis. In addition to Fas1 the initial reactions of fatty acid synthesis are driven by acetyl-CoA carboxylase, which catalyzes the formation of malonyl-CoA. In S. cerevisiae the ACC1 gene encodes this activity. The activity of Acc1 is negatively regulated by phosphorylation catalyzed by the Snf1 protein kinase [32]. With the intent of reducing Acc1 inhibition we deleted the SNF1 gene. However, deletion of SNF1 did not yield and increase in fatty alcohol accumulation. Indeed we repeatedly observed reduced free fatty alcohol concentrations in snf111 strains (data not shown). This may be the result of complex activities of Snf1 in metabolic regulation including the transcriptional down regulation of ACC1 and FAS1 in snf1 mutants [33].

Reduced Degradation of Acyl-CoA Species Increases Fatty Alcohol Accumulation in Engineered Strains.

In S. cerevisiae, fatty acids can be subject to a variety of metabolic fates dependent upon the cells requirements. In addition to incorporation into membranes and storage as triacylglycerides, fatty acids can be degraded by β-oxidation to yield acetyl-CoA. Typically excess fatty acids would be activated to acyl-CoA molecules by an acyl-CoA synthetase. Acyl-CoA molecules act as substrate for fatty acyl-CoA oxidase, an enzyme encoded by POX1 in S. cerevisiae and the first step in the β-oxidation processes. Deletion of POX1 in a strain over expressing FAS1 and harboring the pALC195m plasmid yielded a significant increase in the accumulation of hexadecanol and octadecanol. 50 mL shake flask fermentation experiments with two independently constructed strains revealed that POX1 deletion yielded an increase in hexadecanol from 0.16 mg/g cells to 0.26 mg/g cells. Additionally, octadecanol concentration increased from 0.03 mg/g cells to 0.33 mg/g cells (FIG. 4B). The pox1 mutation caused no reduction in cell viability when glucose was available as a carbon source and caused a very modest reduction in growth rate in synthetic medium, however the POX1 and pox111 cultures reached similar cell densities over the 48 hour fermentation experiment (data not shown).

Analysis of fatty acids in these strains revealed a 60% reduction in the abundance of C16:0, 75% reduction in C16:1, a 6% increase in C18:0 and a 24% reduction in C18:1 (FIG. 4C). The reduction in fatty acids with a commensurate increase in fatty alcohol suggested that fatty acids were being converted to alcohols. Notably the abundance of fatty acids remained greater than the accumulated fatty alcohols suggesting that the available fatty acid pool was not limiting to hexadecanol and octadecanol production.

Reduced Nitrogen Increases Production of Fatty Alcohols.

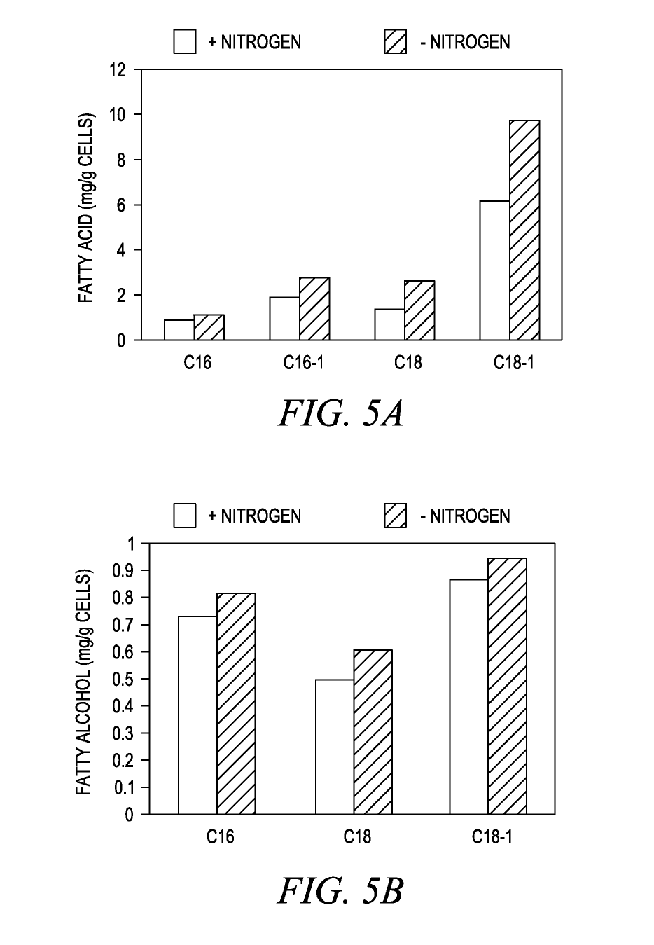

Growth under low nitrogen conditions increases that fatty acid accumulation in many cell types by forcing cells to scavenge nitrogen from amino acids. This can potentially lead to a reduction in citric acid cycle intermediates and result in accumulation of citrate, which stimulates fatty acid synthesis. We tested the hypothesis that reduced nitrogen availability would increase fatty alcohol synthesis in our recombinant S. cerevisiae strains. Cells harboring plasmid pALC195m, cultured in growth medium lacking ammonium sulfate grew to lower cell density than cells cultured in the presence of ammonium sulfate (OD600 3.4 compared to OD600 5.4 with ammonium sulfate) but accumulated higher concentrations of all the measured fatty acids (FIG. 5A). C16:0 increased by 27%, C16:1 increased by 48%, C18:0 increased by 91%, and C18:1 increased by 56%. A smaller increase was observed in the concentration of fatty alcohol under nitrogen limiting growth conditions. Hexadecanol increased by 11% octadecanol increased by 20%, and (Z)-9-octadecanol increased by 8% (FIG. 5B).

Production of Hexadecanol and Octadecanol in Fed Batch S. cerevisiae Fermentation.

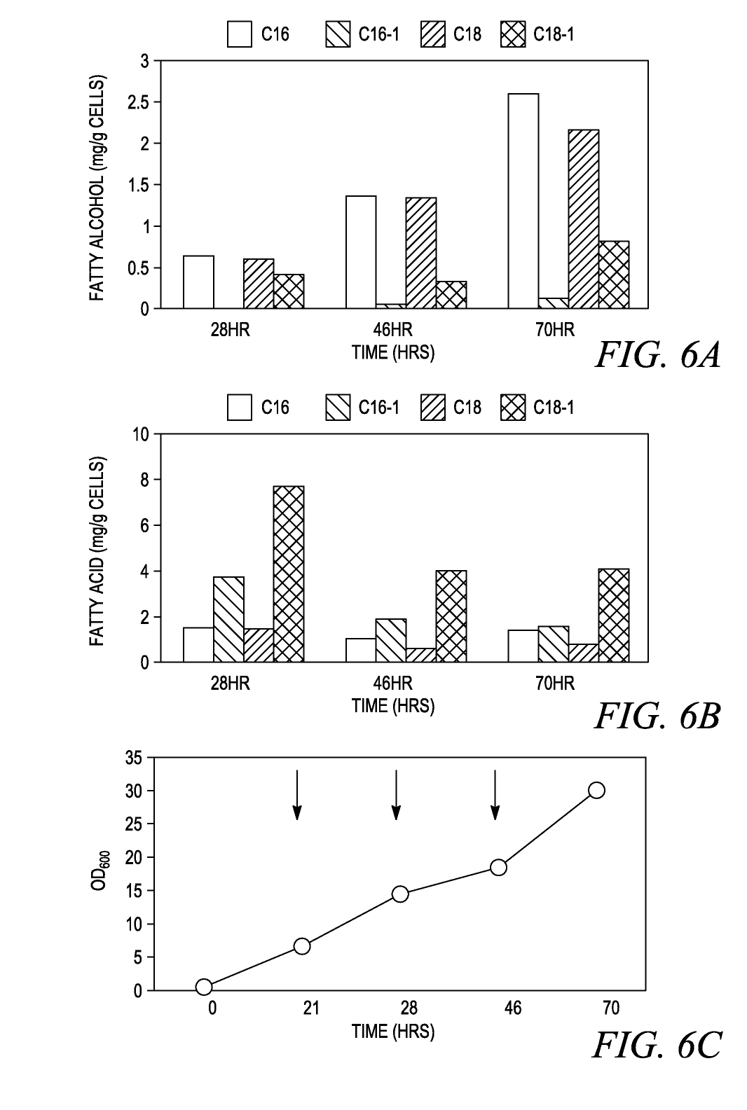

To investigate the potential for fatty alcohol production by S. cerevisiae on a larger scale we performed a fed-batch fermentation trial on a 4 L scale with strain DSY2003 harboring pALC195m. This trial was performed in rich glucose medium rather than synthetic medium. During progression of the fermentation we observed an increase in fatty alcohol accumulation. Over 72 hours the strain ultimately produced 88.4 mg/L hexadecanol, 4.1 mg/L (Z)-9-hexadecanol, 73.5 mg/L octadecanol, and 28.0 mg/L (Z)-9-octadecanol for a combined yield of 194 mg/L C16/C18 alcohol (FIG. 6A). This corresponds to 2.85 mg/g wet weight of cells at a production rate of 2.75 mg/L/hr, and approximately 0.002 g C16/C18/gram glucose. In contrast, fatty acid pools displayed a reduction in abundance as the fermentation progressed consistent with the utilization of nascent fatty acids for the synthesis of fatty alcohol (FIG. 6B). It appeared that fatty alcohol synthesis continued even in the presence of reduced fatty acid pools implying that fatty acid availability had not become limiting within the time frame of this fermentation. Additionally, we did not observe a plateau in biomass accumulation during this experiment suggesting that even greater yields of fatty alcohol could have been achieved without the further addition of sugar (FIG. 6C). The yields of hexadecanol and octadecanol from this strain were greater than those previously reported for S. cerevisiae [27].

Discussion:

Fatty acid based biofuels and oleochemicals are in great demand globally and the market for these compounds continues to grow [34]. The expansion of plantations for palm and oilseed crops has impinged upon rainforest and other habitats as well as creating conflict with food crops [19]. These considerations lead us to investigate the potential for microbial synthesis of fatty alcohols from carbohydrate sources. An additional benefit from a microbial system for fatty alcohol synthesis is the potential to engineer production of molecules with specific chain lengths or modifications.

Fatty alcohol biosynthesis in S. cerevisiae has been recently reported [27]. In that study fatty alcohol yields of approximately 100 mg/L were reported for cells cultured in minimal medium and induced with galactose over 168 hours. The approach reported in this manuscript achieved yields of 194 mg/L of hexadecanol and octadecanol in 72 hours with cells grown in rich medium. The higher production rate observed in our approach may be attributable to the culture conditions in addition to our use of an FAA3 over expressing strain and deletion of the POX1 gene to promote accumulation of acyl-CoA.

Fatty alcohols have also been produced in E. coli where yields of up to 900 mg/L have been reported for cells cultured in minimal medium and induced with arabinose and IPTG [22]. Although yields in that system were high it also made use of inducing agents, arabinose and IPTG, which would significantly add to the cost of any large-scale industrial applications.

We achieved a greater production of fatty acids through the expression of the E. coli thioesterase tesA than with the S. cerevisiae TES1. That tesA is not native to S. cerevisiae may have the advantage that it is not subject to the same forms of regulation imposed upon the endogenous TES1 and thus it may have higher or at least less regulated activity in the cells.

We selected murine FAR1 for a fatty acyl reductase activity based upon its preference for the synthesis of C16 and C18 fatty alcohols. Our experiment made use of the native FAR1 sequence and based upon Western blot analysis the protein was effectively produced in yeast. Codon optimization of the FAR1 gene might improve its production in S. cerevisiae and potentially increase the fatty alcohol production. The Euglena gracilis FAR1 was also produced in S. cerevisiae but displayed much lower activity than murine Far1. It is currently unclear why the Euglena enzyme had less activity in S. cerevisiae. Its activity may have been limited owing to localization or some other aspect of the foreign cellular environment in which it was expressed.

Interestingly, our strains predominantly produced saturated fatty alcohol despite the fact that monounsaturated C16:1 and C18:1 were the most abundant forms of fatty acid in these cells. This likely reflects the substrate preference of the FAR enzyme used in the system. Alternatively it may be that desaturases have little affinity for fatty alcohols or perhaps the fatty alcohols produced do not have access to the endogenous desaturase enzymes. Since FAR enzyme specificity is a likely determinant of the nature of the fatty alcohols produced [11], this opens the possibility that FAR enzymes and thioesterases with different specificities could be employed to tailor a strain as a platform for the synthesis of particular species of fatty alcohols.

An economically viable system for the microbial production of fatty alcohols will be largely dependent upon generating strains that can produce fatty alcohols at close to the theoretical maximum yield of approximately 0.3 g/g glucose [35]. This will likely require further manipulation of the fatty acid biosynthesis pathway in addition to optimization of FAR activity either through identification of a more active enzyme or engineering to increase catalytic activity.

An additional consideration for an economically viable microbial platform for fatty alcohol production is the feedstock used. Feedstock makes up a high proportion of production costs and while most microbial systems will use refined glucose the cost of this may be prohibitive in addition to being a potential conflict with food resources. The sugars derived from lignocellulosic hydrolysates have potential to be the most economical without making use of food grade starch or sugar for the production of industrial chemicals. Yeast is capable of fermenting the sugars from relatively crude hydrolysates and strains resistant to the most common inhibitory compounds present in lignocellulosic hydrolysates have been developed [36, 37]. In addition, yeast strains that can ferment xylose as well as glucose have been developed that would allow maximal use of the available carbon in hydrolysates to be utilized [38]. Thus, the robust nature of yeast may allow this organism to be successfully employed as a factory for fatty alcohol production on an industrial scale.

Conclusions:

Fatty alcohols are used in a broad variety of synthetic compounds ranging from detergents, surfactants, and lubricants to cosmetics and pharmaceuticals. The majority of the world's hexadecanol is synthesized from palm oil and as global demand increases the expansion of palm plantations has begun to have a profound impact upon the environment and biodiversity in some Asian countries. Additionally, the demand for fatty alcohols influences the price and availability of palm oil for food purposes. We have been able to achieve high-level production of hexadecanol and octadecanol in S. cerevisiae. This initial demonstration of the potential to use yeast as a production platform has yet to be optimized and thus has the potential for far greater yields. Yeast has several advantages as a platform for hexadecanol and octadecanol synthesis. Unlike the situation in E. coli hexadecanol and octadecanol are the primary fatty alcohols produced in yeast. Since little C12/C14 alcohols are present in yeast this simplifies the separation and downstream processing for the final product. Additionally, yeast is very robust and performs well under industrial scale fermentation conditions offering the potential to produce fatty alcohols from cellulosic hydrolysates.

Methods:

Yeast Strains, Bacteria Strains, Media, and Growth Conditions:

All of the S. cerevisiae strains used in this work were derived from W303 [39]. The relevant genotype of all strains is listed in Table 1. To delete the POX1 and ACB1 genes in W303, the pox1::KanMX4 and acb1::KanMX4 alleles were amplified from a BY4741 strains obtained from the yeast gene deletion collection (open biosystems). The oligonucleotides used to obtain these fragments (pox1 [symbol] 5/pox1 [symbol] 3 and acb1 [symbol] 5/acb1 [symbol] 3) yield DNA fragments with the KanMX4 cassette flanked by 500 bp of POX1 or ACB1 5′ sequence and 500 bp of POX1 or ACB1 3′ DNA sequence. W303 was transformed with these DNA fragment sand pox1::KanMX4 and acb1::KanMX4 disruptants were selected on YEPD medium supplemented with 200 μg/mL G418. The deletions were confirmed by PCR analysis of genomic DNA from candidate strains. The SNF2 gene was deleted and replaced with a DNA cassette encoding the Kluvermyces lactis LEU2 gene using a PCR based method. Yeast strains were routinely maintained and propagated in rich YEPD medium (yeast extract 10 g/L, peptone 20 g/L, adenine 20 mg/L, tryptophan 20 mg/L and dextrose 20 g/L), strains harboring plasmids were maintained and propagated in selective synthetic medium lacking nutrients required for selection of the auxotrophic marker gene carried on the plasmid. In shake flask experiments yeast strains were cultured aerobically in liquid medium at 30° C. with agitation at 200 rpm for the indicated periods of time. Plasmids were constructed and maintained in E. coli DH5α. Bacterial strains were propagated in LB medium (10 g/L tryptone, 10 g/L NaCl, and 5 g/L yeast extract) supplemented with 100 μg/mL ampicillin, or 50 μg/mL kanamycin as required for plasmid selection.

Cellulosic hydrolysates were prepared from ground wheat straw or softwood sawdust. The feedstocks were treated with 2% sulfuric acid and were subjected to autoclaving at 121° C. for 90 minutes. The insoluble material from his treatment was washed briefly with water, made to a 20% slurry and then enzymatically hydrolyzed with cellulase Optimash 25FPU and Stargen 25FPU/mL (Enzyme solutions). The initial hydrolysis was performed at 50° C. for 1 hour prior to reducing the temperature to 30° C. Urea was added to 15 mM final concentration to provide a nitrogen source. 50 mL of hydrolysate in a shake flask was inoculated with 5×10⁸ yeast cells to initiate the fermentation. The fermentation reactions were carried out over 48 hours at 30° C. with shaking at 150 RPM.

Selection of Lipid Overproducing Strains.

A fatty alcohol producing strain DSY2003 harboring the pALC plasmid was grown to a density of 1×10 cells/mL and plated on top of YEPD agar plates. The plates were subjected to UV mediated mutagenesis with 5 uJ/cm. The cells were allowed to recover for 24 hours in the dark and then harvested and mixed into Percoll solution. The gradients were centrifuged at 30,000×g for 1 hour at 20° C. Cells from the uppermost layers of the gradient were plated to agar plates selecting for the pALC plasmid. Colonies on the selective plates were subsequently stained with Nile Red to detect lipid bodies. Candidate over producers were then grown in small cultures and cell extracts were assayed for fatty alcohols by gas chromatography. Mutations were identified by resequencing of the pALC expression plasmid which revealed a truncation. Several of the lipid over producing mutants displayed an inability to utilize sucrose as a carbon source and these were cloned by complementation revealing the SNF2 gene. This mutant isolation strategy was originally described by Kamasaka [41]

Reagents:

Restriction Enzymes, T4 DNA lipase, and Taw DNA polymerase were obtained from New England Bolas. pGEM®-T Easy Vectors were obtained from Promega. DNA purification kits and reagents were supplied by Qiagen. All oligonucleotides used in this study were purchased from Integrated DNA Technologies (IDT). Methanolic HCL, N-methyl-N-(trimethylsiylyl) trifluoroacetamide (MSTFA), HPLC grade hexane and chloroform were from Sigma ALDRICH. All fatty acid and fatty alcohol standards and internal standards used in this study were ordered from Nu-Chek Prep Inc.

Plasmid and Yeast Strain Construction:

The expression vectors used in this study are based on the multicopy plasmid YEpLac195 [42]. The poly linker of YEpLac195 was removed and replaced with a poly linker containing sites for restriction enzymes EcoRI-NotI-XbaI-SpeI-NotI-PstI, this plasmid is referred to as YEplac195bb. A 750 bp DNA fragment from upstream of the PGK1 gene was amplified with oligonucleotides PGK750X and PGK750S. The resulting 750 bp fragment was ligated into the XbaI and SpeI sites of YEplac195bb to yield YEp195-PPGK1. A 400 bp DNA fragment from upstream of the HXT7 gene was amplified with oligonucleotides HXT400X and HXT400S. The resulting 400 bp fragment was ligated into the XbaI and SpeI sites of YEplac195bb to generate YEp195-PHXT7. A 1000 bp DNA fragment from upstream of the PYK1 gene was amplified with oligonucleotides PYK1000X and PYK1000S. The resulting DNA fragment was ligated into the XbaI and SpeI sites of YEplac195bb to generate YEp195-PPYK1. An 830 bp DNA fragment from upstream of the FBA1 gene was amplified with oligonucleotides FBA830X and FBA830S. The resulting DNA fragment was ligated into the XbaI and SpeI sites of YEplac195bb to produce YEp195-PFBA1.

The S. cerevisiae FAS1 and FAA3 genes were obtained by PCR amplification from S. cerevisiae genomic DNA using oligonucleotide primers FAS15bb and FAS13bb, FAA35bb, and FAA33bb. The Mus musculus FAR1 (mFAR1) open reading frame was amplified from mouse total cDNA using oligonucleotides FAR15bb and FAR13bb. The Euglena gracilis FAR1 (egFAR1) open reading frame was amplified from plasmid pPT515 [43]. These oligonucleotides introduce coding sequence for an 11 amino acid epitope recognized by the anti-MYC 9E10 antibody to the 3′ end of mFAR1. The truncated tesA open reading was amplified from E. coli genomic DNA using oligonucleotides TESA5bb, and TESA3bb. TES1 was amplified from yeast genomic DNA using oligonucleotides TES15bb and TES13bb. These oligonucleotides introduce coding sequence for an 11 amino acid epitope recognized by the anti-MYC 9E10 antibody to the 3′ end of tesA and TES1. The PCR amplified DNA fragments were initially ligated with the pGEM-T easy vector. Each gene was sequenced in its entirety. Individual clones that contained no mutations that altered the amino acid sequence were used for subsequent yeast expression plasmid constructions. The restriction enzymes XbaI and PstI were used to excise the FAS1, FAA3, mFAR1, egFAR1, TES1, and tesA open reading frames from pGEM-T. FAS1 was ligated into YEp195-PPGK1 to generate YEp195-PPGK1-FAS1. FAA3 was ligated with YEp195-PHXT7 to create YEp195-PHXT7-FAA3, mFAR1 and egFAR1 were independently ligated with YEp-PPYK1 and then a 250 bp DNA fragment containing the transcriptional termination sequence from the CYC1 gene was ligated into the plasmids 3′ to the FAR1 open reading frames to generate YEp195-PPYK1-mFAR1 and PPYK1-egFAR1. The TES1 and tesA open reading frames were ligated with YEp195-PFBA1 and then a 300 bp fragment containing the transcriptional termination sequence from the S. cerevisiae ADH1 gene was ligated 3′ to tesA or TES1 to generate YEp195-PFBA1-tesA and YEp195-PFBA1-TES1. The PPYK1-FAR1 fusion genes was excised from YEp195-PPYK1-mFAR1 and YEp195-PPYK1-egFAR1 as an XbaI/PstI fragment and ligated into the SpeI/PstI cut YEp195-PHXT7-FAA3 expression plasmid, subsequently the XbaI/PstI PFBA1-tesA fragment was added thus combining FAA3, mFAR1 or egFAR1, and tesA into a single expression plasmid named pALC195m and pALC195eg. The plasmid pRS303-PPGK1-FAS1 was constructed by ligating a 4000 bp XbaI-BamHI PPGK1-FAS1 fragment from YEp195-PPGK1-FAS1 into pRS303 [44]. This plasmid was cleaved with PacI to direct its integration into the endogenous S. cerevisiae FAS1 gene thus placing the chromosomal FAS1 open reading frame under the regulation of the PGK1 promoter, generating the strain fas1::PPGK1-FAS1::HIS3.

Protein Analysis and Enzyme Assays:

Expression of mFar1 egFar1, Tes1 and tesA proteins in S. cerevisiae was confirmed by Western blot analysis. Cultures of yeast harboring the expression plasmids pALC195m, or pALC195eg were collected by centrifugation and samples of cells equivalent to 5 OD600 units were lysed by grinding with glass beads in 20% trichloroacetic acid as described [45]. The whole cell extracts were allowed to precipitate on ice for 20 minutes then collected by centrifugation at 10,000×g for 20 minutes. The protein pellet was washed with 1 mL of ice-cold acetone, dried and resuspended in 1×SDS sample buffer (2% SDS, 62.5 mM, Tris-HCl pH 6.8, 100 mM DTT, 10% glycerol). 50 μg of total protein was separated by gel electrophoresis through a 10% polyacrylamide gel and transferred to a polyvinylidene fluoride (PVDF) membrane. The membranes were blocked for eight hours at 4° C. in a solution of Tris Buffered Saline (25 mM Tris-HCl pH 7.6, 150 mM NaCl, 2 mM KCl) supplemented with 0.25% Tween 20 and 10% nonfat milk powder. Then probed for 8 hours with anti-MYC ascites fluid (Covance laboratories) at a dilution of 1:10,000. Following extensive washing the primary antibodies were detected with Horse Radish Peroxidase labeled anti-mouse secondary antibody (Jackson Laboratories) applied to the blots at a 1:5,000 dilution. The protein signals were visualized with enhanced chemiluminescence.

Extraction and Analysis of Fatty Acid and Fatty Alcohols:

To extract total cellular lipids, 25 mL of culture was harvested when the desired growth phase was achieved (O.D.600 reading 6 to 8 in shake flask experiment, or at the indicated times in fed-batch fermentation experiment). Cells were collected by centrifugation at 3000×g 3 min and washed in sterile water. Cell pellets were weighted and resuspended in 3 mL of methanolic-HCL. The suspensions were transferred into glass tubes and lysed by grinding with glass beads (3×1 min grinding with 1 min off intervals between grinding) and incubated at 80° C. for 2 hours. Fatty acids and fatty alcohols were extracted in 2 mL n-hexane spiked with 0.5 mg or 1 mg of heptadecanoic acid (C17) as an internal standard followed by shaking at 200 rpm for 30 min. The hexane phase was transferred into a fresh glass vial and derivatized with 50 μL MSTFA at 70° C. for 15 min. All extracted samples were dried under N₂ gas and resuspended in 1 mL of chloroform. Samples were stored at −20° C. until gas chromatography (GC) analyses. The fatty acid and fatty alcohol composition of the total lipid samples were analyzed as their derivatized forms. Lipids were analyzed with an Agilent technologies 6890N Network GC System gas chromatography instrument using Flame Ionization Detection. The separation was performed using an HP MS5, 30 m×250 μm×0.25 μm, non-polar capillary column (Agilent Technologies, Santa Clara, Calif.), The GC program was as follows: initial temperature of 70° C., held for 1 min, ramped to 300° C. at 10° C. per min and held for 10 min. Quantitation of fatty alcohol species was performed with a standard curve constructed with 0.1 mg, 1.0 mg, and 10.0 mg of the standards C16:0-OH (hexadecanol), C16:1-OH ((Z)-9-hexadecanol), C18:0-OH (octadecanol), C18:1-OH ((Z)-9-octadecanol) to generate a standard curve to allow accurate quantitation of fatty alcohols by FID.

Fed-Batch Fermentation Culture:

The fas1::PPGK1-FAS1 pox1::KanMX4 pALC195m strain was initially cultured in 500 mL −Ura +dextrose medium in a 2 L flask. When the cell culture reached early stationary phase, 350 mL of culture was transferred into the 5 L Biostat B fermenter, which held 3.5 L of YEPD (yeast extract 10 g/L, peptone 20 g/L, adenine 20 mg/L, tryptophan 40 mg/L, and dextrose 60 g/L). The fermenter temperature was maintained at 30° C. 2 M NaOH and 2 M HCL were added to the fermentation vessel to maintain the pH at 5.6. The aeration rate was 10 L/min. and the stir rate was maintained at 500 rpm. The feeding schedule consisted of a 100 mL aliquot of 100% glucose addition into the culture every 12 hours.

### Tables:

TABLE 1
Saccharomyces cerevisiae strains used in this study

| Strain | Background | Relevant genotype |
|---|---|---|
| W303-1A | W303 | MATa leu2-3,112, trp1-1 can1-100 ura3-1 |
| | | ade2-1 his3-11,15 |
| DSY2001 | W303 | fas1::PPGK1-FAS1-HIS3 |
| DSY2002 | W303 | pox1::KanMX4 |
| DSY2003 | W303 | pox1::KanMX4 fas1::PPGK1-FAS1-HIS3 |
| DSY2004 | W303 | pox1::KanMX4 fas1::PPGK1-FAS1-HIS3 |
| | | snf2::LEU2 |
| DSY2005 | BY4741 | MATa his3Δ1 leu2Δ0 MET15Δ0 ura3Δ0 |
| DSY2006 | BY4741 | acb1::KanMX4 |

TABLE 2
Plasmids used in this study

| Plasmid | Relevant genotype |
|---|---|
| YEplacl95bb | YEplac195 with modified polylinker URA3 selection |
| pFBA1-TESA | YEplac195 FBA1 promoter driving tesA, cyc1 |
| terminator | |
| pFBA1-TES1 | YEplac195 FBA1 promoter driving TES1, cyc1 |
| terminator | |
| pPYK1-mFAR1 | YEplac195 PYK1 promoter driving mFAR1, ADH1 |
| | terminator |
| pPYK1-egFAR1 | YEplac195 PYK1 promoter driving egFAR1, ADH1 |
| terminator | |
| pHXT7-FAA3 | YEplac195 HXT7 promoter driving FAA3 |
| pALC195m | YEplac195 HXT7-FAA3 PYK1-mFAR1 FBA1-tesA |
| pALC195eg | YEplac195 HXT7-FAA3 PYK1-egFAR1 FBA1-tesA |
| pPGK1-FAS1 | pRS303 PGK1 promoter driving truncated FAS1, |
| | HIS3 selection |

TABLE 3
Oligonucleotide primers used in this study

| Oligonu- | |
|---|---|
| cleotide | Sequence 5′-3′ |
| Pox1115 | TCTTTGTATTTATTCCTAGCG |
| Pox1113 | GATTGTTACCATAGCAACTCA |
| PGK750X | CACGTTCTAGAACGCACAGATATTATAACATCTG |
| PGK750S | CAGGTACTAGTTGTTTTATATTTGTTGTAAAAAGTAG |
| HXT400X | CACGTTCTAGACCACTACTTCTCGTAGGAAC |
| HXT400S | CAGGTACTAGTTTTTTGATTAAAATTAAAAAAACTTTTT |
| PYK1000X | CACGTTCTAGAAATGCTACTATTTTGGAGATTAAT |
| PYK1000S | CAGGTACTAGTTGTGATGATGTTTTATTTGTTTTG |
| FBA830X | CACGTTCTAGAAGTTGATGGATCCAACTGGC |
| FBA830S | CAGGTACTAGTTTTGAATATGTATTACTTGGTTATG |
| FAS15bb | CTTCTAGAATGGACGCTTACTCCACAAGACCATTA |
| FAS13bb | GCTGCAGCGGCCGCACTAGTCTAAAGGTCCTCCTCAGAG |
| | ATTAATTTCTGTTCGGATTGTTCATACTTTTCCCAG |
| FAA5bb | TGTAGAATGTCCGAACAACACTCTGTC |
| FAA3bb | GCTGCAGCGGCCGCACTAGTCAATAGCAAAATGTGATTTC |
| mFAR5bb | TCTAGAATGGTTTCAATCCCAGAATAC |
| mFAR3bb | GCTGCAGCGGCCGCACTATGCTAAAGGTCCTCCTCAGAG |
| | ATTAATTTCTGTTCGTATCTCATAGTGCTGGATGC |
| egFAR5bb | CTTCTAGAATGAACGATTTCTACGCG |
| egFAR3bb | GCTGCAGCGGCCGCACTAGTCTAAAGGTCCTCCTCAGAG |
| | ATTAATTTCTGTTCCAGCATGGCCCGCAGGAATC |
| TESA5bb | GTTCTAGAATGGCGGACACGTTATTGATTCTG |
| TESA3bb | CACTAGTCTAAAGGTCCTCCTCAGAGATTAATTTCTGTT |
| | CTGAGTCATGATTTACTAAAGG |
| TES15bb | GCTTCTAGAATGAGTGCTTCCAAAATGGC |
| TES13bb | CTACTAGTCTACAGGTCCTCCTCAGAGATTAATTTCTGT |
| | TCGAACTTGGCTCGAATGT |

TABLE 4
Hexadecanol production by engineered S. cerevisiae

| Vector | Hexadecanol | | Octadecanol (mg/L culture) | |
|---|---|---|---|---|
| Vector | 0.00 | 0.00 | 0.00 | 0.00 |
| pALCeg | 0.01 | 0.01 | 0.00 | 0.00 |
| pALCm | 1.64 | 1.72 | 0.68 | 0.67 |

Values were obtained from two independent 48 hour fermentations.

### CITATIONS

1. Tillman J A, Seybold S J, Jurenka R A, Blomquist G J: Insect pheromones—an overview of biosynthesis and endocrine regulation. Insect Biochem Mol Biol 1999, 29(6):481-514.
2. Metz J G, Pollard M R, Anderson L, Hayes T R, Lassner M W: Purification of a jojoba embryo fatty acyl-coenzyme A reductase and expression of its cDNA in high erucic acid rapeseed. Plant Physiol 2000, 122(3):635-644.
3. Lienard M A, Hagstrom A K, Lassance J M, Lofstedt C: Evolution of multicomponent pheromone signals in small ermine moths involves a single fatty-acyl reductase gene. Proc Natl Acad Sci USA 2010, 107(24):10955-10960.
4. Fixter L M, Nagi N M, McCormak J G, Fewson C A: Structure, distribution and function of wax esters in Acinetobacter calcoaceticus. Microbiology 1986, 132:3147-3157.
5. Tucci S, Vacula R, Krajcovic J, Proksch P, Martin W: Variability of wax ester fermentation in natural and bleached Euglena gracilis Strains in response to oxygen and the elongase inhibitor flufenacet. J Eukaryot Microbiol 2010, 57(1):63-69.
6. Ju S J, Harvey H R: Lipids as markers of nutritional condition and diet in the Antarctic krill Euphausia superba and Euphausia crystallorophias during austral winter. Deep-Sea Research Part II-Topical Studies in Oceanography 2004, 51:17-19.
7. Kolattukudy P E: Reduction of fatty acids to alcohols by cell-free preparations of Euglena gracilis. Biochemistry 1970, 9(5):1095-1102.
8. Pollard M R, McKeon T, Gupta L M, Stumpf P K: Studies on biosynthesis of waxes by developing jojoba seed. II. The demonstration of wax biosynthesis by cell-free homogenates. Lipids 1979, 14:651-662.
9. Wang X, Kolattukudy P E: Solubilization and purification of aldehyde-generating fatty acyl-CoA reductase from green alga Botryococcus braunii. FEBS Lett 1995, 370(1-2):15-18.
10. Teerawanichpan P, Qiu X: Molecular and functional analysis of three fatty acyl-CoA reductases with distinct substrate specificities in copepod Calanus finmarchicus. Mar Biotechnol (NY) 2012, 14(2):227-236.
11. Domergue F, Vishwanath S J, Joubes J, Ono J, Lee J A, Bourdon M, et al.: Three Arabidopsis fatty acyl-coenzyme A reductases, FAR1, FAR4, and FAR5, generate primary fatty alcohols associated with suberin deposition. Plant Physiol 2010, 153(4):1539-1554.
12. Vioque J, Kolattukudy P E: Resolution and purification of an aldehyde-generating and an alcohol-generating fatty acyl-CoA reductase from pea leaves (Pisum sativum L.). Arch Biochem Biophys 1997, 340(1):64-72.
13. Rowland O, Domergue F: Plant fatty acyl reductases: enzymes generating fatty alcohols for protective layers with potential for industrial applications. Plant Sci 2012, 193-194:28-38.
14. Cheng J B, Russell D W: Mammalian wax biosynthesis. I. Identification of two fatty acyl-Coenzyme A reductases with different substrate specificities and tissue distributions. J Biol Chem 2004, 279(36):37789-37797.
15. Knaut J, Richtler H J: Trends in industrial uses of palm and lauric oils. J Amer Oil Chem Soc 1985, 62:317-327.
16. Khalkar S, Bhowmick D, Pratap A: Synthesis of polymers from fatty alcohol and acrylic acid and its impact on tribological properties. J Oleo Sci 2013, 62(3):167-173.
17. Rupilius W, Ahmad S: Palm oil and palm kernel oil as raw materials for basic oleochemicals and biodiesel. European Journal of lipid science and technology 2007, 109:433-439.
18. Carter C, Finley W, Fry J, Jackson D, Willis L: Palm oil markets and future supply. European Journal of Lipid Science and Technology 2007, 109(4):307-314.
19. Fitzherbert E B, Struebig M J, Morel A, Danielsen F, Bruhl C A, Donald P F, et al.: How will oil palm expansion affect biodiversity?Trends Ecol Evol 2008, 23(10):538-545.
20. Lees A C, Vieira I C: Forests: Oil-palm concerns in Brazilian Amazon. Nature 2013, 497:188.
21. Zheng Y N, Li L L, Liu Q, Yang J M, Wang X W, Liu W, et al.: Optimization of fatty alcohol biosynthesis pathway for selectively enhanced production of C12/14 and C16/18 fatty alcohols in engineered Escherichia coli. Microb Cell Fact 2012, 11:65.
22. Liu A, Tan X, Yao L, Lu X: Fatty alcohol production in engineered E. coli expressing Marinobacter fatty acyl-CoA reductases. Appl Microbiol Biotechnol 2013, 97(15):7061-7071.
23. Tan X, Yao L, Gao Q, Wang W, Qi F, Lu X: Photosynthesis driven conversion of carbon dioxide to fatty alcohols and hydrocarbons in cyanobacteria. Metab Eng 2011, 13(2):169-176.
24. Akhtar M K, Turner N J, Jones P R: Carboxylic acid reductase is a versatile enzyme for the conversion of fatty acids into fuels and chemical commodities. Proc Natl Acad Sci USA 2013, 110(1):87-92.
25. Hong K K, Nielsen J: Metabolic engineering of Saccharomyces cerevisiae: a key cell factory platform for future biorefineries. Cell Mol Life Sci 2012, 69(16):2671-2690.
26. Ageitos J M, Vallejo J A, Veiga-Crespo P, Villa T G: Oily yeasts as oleaginous cell factories. Appl Microbiol Biotechnol 2011, 90(4):1219-1227.
27. Runguphan W, Keasling J D: Metabolic Engineering of Saccharomyces cerevisiae for Production of Fatty Acid-Derived Biofuels and Chemicals. Metab Eng 2013:ePub ahead of print.
28. Cho H, Cronan J E, Jr.: Defective export of a periplasmic enzyme disrupts regulation of fatty acid synthesis. J Biol Chem 1995, 270(9):4216-4219.
29. Wenz P, Schwank S, Hoja U, Schuller H J: A downstream regulatory element located within the coding sequence mediates autoregulated expression of the yeast fatty acid synthase gene FAS2 by the FAS1 gene product. Nucleic Acids Res 2001, 29(22):4625-4632.
30. Schuller H J, Hahn A, Troster F, Schutz A, Schweizer E: Coordinate genetic control of yeast fatty acid synthase genes FAS1 and FAS2 by an upstream activation site common to genes involved in membrane lipid biosynthesis. EMBO J 1992, 11(1):107-114.
31. Chirala S S: Coordinated regulation and inositol-mediated and fatty acid-mediated repression of fatty acid synthase genes in Saccharomyces cerevisiae. Proc Natl Acad Sci USA 1992, 89(21):10232-10236.
32. Woods A, Munday M R, Scott J, Yang X, Carlson M, Carling D: Yeast SNF1 is functionally related to mammalian AMP-activated protein kinase and regulates acetyl-CoA carboxylase in vivo. J Biol Chem 1994, 269(30):19509-19515.
33. Usaite R, Jewett M C, Oliveira A P, Yates J R, 3rd, Olsson L, Nielsen J: Reconstruction of the yeast Snf1 kinase regulatory network reveals its role as a global energy regulator. Mol Syst Biol 2009, 5:319.
34. Metzger J O, Bornscheuer U: Lipids as renewable resources: current state of chemical and biotechnological conversion and diversification. Appl Microbiol Biot 2006, 71(1): 13-22.
35. Lennen R M, Pfleger B F: Engineering Escherichia coli to synthesize free fatty acids. Trends Biotechnol 2012, 30(12):659-667.
36. Koppram R, Albers E, Olsson L: Evolutionary engineering strategies to enhance tolerance of xylose utilizing recombinant yeast to inhibitors derived from spruce biomass. Biotechnol Biofuels 2012, 5(1):32.
37. Weber C, Farwick A, Benisch F, Brat D, Dietz H, Subtil T, et al.: Trends and challenges in the microbial production of lignocellulosic bioalcohol fuels. Appl Microbiol Biotechnol 2010, 87(4):1303-1315.
38. Cai Z, Zhang B, Li Y: Engineering Saccharomyces cerevisiae for efficient anaerobic xylose fermentation: reflections and perspectives. Biotechnol J 2012, 7(1):34-46.
39. Thomas B J, Rothstein R: Elevated recombination rates in transcriptionally active DNA. Cell 1989, 56(4):619-630.
40. Sherman F: Getting started with yeast. Methods Enzymol 2002, 350:3-41.
41. Kamisaka Y, Noda N, Tomita N, Kimura K, Kodaki T, Hosaka K: Identification of genes affecting lipid content using transposon mutagenesis in Saccharomyces cerevisiae. Biosci Biotechnol Biochem 2006, 70:646-653.
42. Gietz R D, Sugino A: New yeast-Escherichia coli shuttle vectors constructed with in vitro mutagenized yeast genes lacking six-base pair restriction sites. Gene 1988, 74(2):527-534.
43. Teerawanichpan P, Qiu X: Fatty acyl-CoA reductase and wax synthase from Euglena gracilis in the biosynthesis of medium-chain wax esters. Lipids 2010, 45(3):263-273.
44. Sikorski R S, Hieter P: A system of shuttle vectors and yeast host strains designed for efficient manipulation of DNA in Saccharomyces cerevisiae. Genetics 1989, 122(1):19-27.
45. Foiani M, Liberi G, Lucchini G, Plevani P: Cell cycle-dependent phosphorylation and dephosphorylation of the yeast DNA polymerase alpha-primase B subunit. Mol Cell Biol 1995, 15(2):883-891.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An isolated Saccharomyces cerevisiae cell genetically engineered to produce one or more fatty alcohols, the S. cerevisiae cell genetically engineered to express or overexpress two or more enzymes involved in fatty alcohol synthesis and further genetically engineered to be deficient in at least one enzyme involved in fatty acid oxidation.

2. The isolated S. cerevisiae cell according to claim 1, wherein the one or more fatty alcohols is octadecanol or hexadecanol.

3. The isolated S. cerevisiae cell according to claim 1, wherein the two or more enzymes involved in fatty alcohol synthesis comprise acyl-CoA thioesterase and fatty acyl reductase.

4. The isolated S. cerevisiae cell according to claim 3, wherein the acyl-CoA thioesterase is an E. coli acyl-CoA thioesterase.

5. The isolated S. cerevisiae cell according to claim 3, wherein the fatty acyl reductase is a Mus musculus fatty acyl reductase.

6. The isolated S. cerevisiae cell according to claim 3, wherein the two or more enzymes involved in fatty alcohol synthesis further comprise a fatty acyl activating enzyme.

7. The isolated S. cerevisiae cell according to claim 6, wherein the fatty acyl activating enzyme is a S. cerevisiae fatty acyl activating enzyme.

8. The isolated S. cerevisiae cell according to claim 7, wherein the acyl-CoA thioesterase, fatty acyl reductase and fatty acyl activating enzyme are expressed from a vector comprising a first nucleic acid encoding the acyl-CoA thioesterase, a second nucleic acid encoding the fatty acyl reductase and a third nucleic acid encoding the fatty acyl activating enzyme.

9. The isolated S. cerevisiae cell according to claim 3, wherein the two or more enzymes involved in fatty alcohol synthesis further comprise fatty acyl synthetase.

10. The isolated S. cerevisiae cell according to claim 9, wherein the fatty acyl synthetase is an endogenous fatty acyl synthetase.

11. The isolated S. cerevisiae cell according to claim 1, wherein the at least one enzyme involved in fatty acid oxidation comprises fatty acyl-CoA oxidase.

12. An isolated Saccharomyces cerevisiae cell genetically engineered to produce one or more fatty alcohols, the S. cerevisiae cell genetically engineered to:
i) express an acyl-CoA thioesterase;
ii) express a fatty acyl reductase, and
iii) (a) express a fatty acyl activating enzyme, or
(b) lack a fatty acyl-CoA oxidase, or
(c) both (a) and (b).

13. The isolated S. cerevisiae cell according to claim 12, wherein the one or more fatty alcohols is octadecanol or hexadecanol.

14. The isolated S. cerevisiae cell according to claim 12, wherein the acyl-CoA thioesterase is an E. coli acyl-CoA thioesterase.

15. The isolated S. cerevisiae cell according to claim 12, wherein the fatty acyl reductase is a Mus musculus fatty acyl reductase.

16. The isolated S. cerevisiae cell according to claim 12, wherein the S. cerevisiae cell is genetically engineered to express a fatty acyl activating enzyme.

17.-21. (canceled)

22. The isolated Saccharomyces cerevisiae cell according to claim 1 wherein the cell is genetically engineered to produce one or more fatty alcohols, the S. cerevisiae cell genetically engineered to express an acyl-CoA thioesterase from E. coli, a fatty acyl reductase from Mus musculus and an endogenous fatty acyl activating enzyme, to over-express an endogenous fatty acyl synthetase and to lack an endogenous fatty acyl-CoA oxidase.

23. (canceled)

24. An isolated nucleic acid comprising a first nucleotide sequence encoding an acyl-CoA thioesterase, a second nucleotide sequence encoding a fatty acyl reductase and a third nucleotide sequence encoding a fatty acyl activating enzyme.

25.-33. (canceled)

34. A method of producing one or more fatty alcohols comprising culturing the isolated S. cerevisiae cell of claim 1 and isolating the one or more fatty alcohols.

35. A method of producing fatty alcohols comprising hexadecanol and octadecanol, the method culturing the isolated S. cerevisiae cell of claim 28 in a medium comprising glucose and isolating the one or more fatty alcohols.

36.-41. (canceled)
